(19) United States
(12) Patent Application Publication
(10) Pub. No.: US 2013/0287803 A1
(43) Pub. Date: Oct. 31, 2013

(54) NOVEL USES OF VEGFXXXB

(71) Applicant: The University of Bristol, Clifton (GB)

(72) Inventors: David Bates, Clifton (GB); Lucy Donaldson, Clifton (GB); Steven James Harper, Clifton (GB); Jing Hua, Clifton (GB)

(21) Appl. No.: 13/874,173

(22) Filed: Apr. 30, 2013

Related U.S. Application Data
(63) Continuation of application No. 12/918,363, filed on Nov. 3, 2010, now abandoned.

(30) Foreign Application Priority Data
Feb. 29, 2008 (GB) GB0803912.5

(51) Int. Cl. A61K 38/18; A61K 45/06; C12N 15/85

(52) U.S. Cl. CPC A61K 38/1866; C12N 15/85; A61K 45/06
USPC 424/184.1; 435/375; 435/455; 514/44 R; 424/94.5

(57) ABSTRACT

The invention provides VEGFₓₓₓb, or an agent which selectively promotes the expression of VEGFₓₓₓb in preference to VEGFₓₓₓ in cells of a subject or in vitro, or an expression vector system which causes the expression of the VEGFₓₓₓb in a host organism, for use in treating or preventing neuropathic and neurodegenerative disorders, or for use as a neuroprotective or neuroregenerative agent in vivo or in vitro.

The VEGFₓₓₓb is preferably VEGF₁₆₅b.

### CROSS-REFERENCE TO RELATED APPLICATIONS

This is a continuation application of U.S. application Ser. No. 12/918,363, filed Nov. 3, 2010, which claims priority to International Serial No. PCT/GB2009/000571, filed Feb. 27, 2009 which claims priority of British Patent Application No. 0803912.5, filed Feb. 29, 2008. The entire disclosures of the applications identified in this paragraph are incorporated herein by references.

### FIELD OF THE INVENTION

The present invention relates to novel uses of VEGFₓₓₓb, particularly as a neuroprotective or neuroregenerative agent.

The present invention also relates to corresponding uses of agents that promote the endogenous expression of VEGFₓₓₓb by the subject in need of neuroprotection or neuroregeneration via alternative splicing of the VEGF gene.

### BACKGROUND OF THE INVENTION

A family of alternative splice variants of vascular endothelial growth factor (VEGF) has recently been identified ((WO-A-03/012105; Bates et al, Cancer Res. 62, 4123-4131 (2002); Bates et al, Clinical Science 110, 575-585 (2006)).

These variants are formed by splicing from exon 7 into the previously assumed 3′ UTR of the VEGF mRNA, forming proteins of the same length as other forms, but with a different C terminal amino acid sequence. This family of splice variants has been termed “VEGFₓₓₓb”, where “xxx” refers to the number of amino acids in the isoform, e.g. 165 in the case of VEGF₁₆₅b. We have identified VEGF₁₆₅b, VEGF₁₈₉b, VEGF₁₄₅b, VEGF₁₈₃b and VEGF₁₂₁b proteins (O. Konopatskaya, Molecular Vision. 2006; 12:626-632).

Interestingly, the VEGFR2 (VEGF Receptor 2) and NP-1 (Neuropilin-1) binding domains and dimerisation domains are intact in these newly identified isoforms, although the new C-terminal sequence abuts the NP-1 binding site and appears to interfere with NP-1 binding (Cebe Suarez 2006; Cell Mol Life Sci. 2006 September; 63(17):2067-77).

VEGF₁₆₅b inhibits the endothelial proliferative, migratory and vasodilator effects of VEGF₁₆₅, was anti-angiogenic in the rabbit cornea, mouse retina and subcutaneous tissue, and the rat mesentery, and inhibited tumour growth in xenotransplanted tumours in mice (O. Konopatskaya, Molecular Vision. 2006; 12:626-632) (Cebe Suarez 2006; Cell Mol Life Sci. 2006 September; 63(17):2067-77) (Woolard J., Cancer Res 64: 7822-7835, 2004).

VEGF, acting through its receptor (VEGFR2), is the principal agent responsible for blood vessel growth in physiological and pathological angiogenesis (Carmeliet P. Nat Med. 2003 June; 9(6):653-60. Review). It is required for the recovery of tissues after wounding as part of the healing response (Bates D O Int J Low Extreme Wounds. 2003 June; 2(2):107-20), and also for the development of many pathologies, including cancer (Ferrara N. Nat Rev Cancer. 2002 October; 2(10):795-803), diabetes (Chou E Circulation 2002 Jan. 22; 105(3):373-9) and atherosclerosis (Celletti F L Nat Med. 2001 April; 7(4):425-9). It has become clear recently that the action of VEGF is not restricted to the vascular system, but that VEGF is a key component of neuronal protective mechanisms (Jin K L et al Neuroscience. 2000; 99(3):577-85) (Jin K L et al Proc Natl Acad Sci USA. 2000 Aug. 29; 97(18):10242-7) (Oosthuyse et al Nat Genet., 2001 June; 28(2):131-8). This was first identified in vivo through the role of VEGF in preventing progression of the motor neuron disease, amyotrophic lateral sclerosis, by preventing motor neuron cell death (Oosthuyse et al, Nat Genet., 2001 June; 28(2):131-8). There is also a substantial body of evidence to suggest that VEGF may be involved in sensory neuropathy.

VEGF is expressed at low levels in adult dorsal root ganglion (DRG) under normal conditions, yet is detectable in up to 35% of neurons, mainly in small (<30 μm diameter) DRG neurons, although there are no reports on the modality of sensory neuron this population represents. VEGFR2 is also normally expressed in the DRG (<10% of DRG neurons). It is unclear which specific subsets of neurons express VEGFR2 but it is thought that the receptor is not co-expressed with VEGF (Sondell M. Neuroreport. 2001 Jan. 22; 12(1):105-8).

Neurotrophic factors have potentially valuable roles to play in treatment and management of neuropathic and neurodegenerative conditions, and in cognitive stimulants for non-medical use by healthy persons to improve brain function on a temporary basis.

The present invention is based on our unanticipated finding that VEGFₓₓₓb, has neuroprotective and neuroregenerative effects, from which activity is predicted against a range of neuropathic and neurodegenerative conditions, and as a cognitive stimulant for non-medical use by healthy persons to improve brain function on a temporary basis. This finding is surprising in view of the known anti-angiogenic effects of VEGFₓₓₓb and the knowledge that, as mentioned above, VEGFₓₓₓb does not bind NP-1.

In WO-A-2008/110777, the disclosure of which is incorporated herein by reference, claiming priority from British Patent Application No. 0704678.2, we describe and claim agents for selectively promoting the expression of VEGFₓₓₓb in preference to VEGFₓₓₓ in cells of a subject or in vitro.

### BRIEF DESCRIPTION OF THE INVENTION

According to a first aspect of the present invention, there is provided VEGFₓₓₓb for use in treating or preventing neuropathic and neurodegenerative disorders, either via neuronal survival/regeneration directly or via cytoprotection of associated or supportive cells (eg glial cells), or for use as a neuroprotective or neuroregenerative agent in vivo or in vitro (including ex vivo).

According to a second aspect of the present invention, there is provided a method of treating or preventing neuropathic and neurodegenerative disorders, or of obtaining neuroprotection or neuroregeneration in vivo or in vitro (including ex vivo), which comprises administering to neurons, in vivo or in vitro (including ex vivo), an effective amount of VEGFₓₓₓb.

According to a third aspect of the present invention, there is provided the use of VEGFₓₓₓb in the manufacture of a composition (e.g. a pharmaceutical composition, foodstuff, food supplement, beverage or beverage supplement) for treating or preventing neuropathic and neurodegenerative disorders, or for obtaining neuroprotection or neuroregeneration in vivo or in vitro (including ex vivo).

The present invention also includes the use of an agent, such as those described and claimed in WO-A-2008/110777, which selectively promotes the expression of VEGFₓₓₓb in preference to VEGFₓₓₓ in cells of a subject or in vitro. The use of such an agent constitutes a further aspect of the present invention. In particular, there may be mentioned agents that favour distal splice site (DSS) splicing during processing of VEGF pre-mRNA transcribed from the C terminal exon 8 of the VEGF gene. Such agents may, if desired be used in association with one or more controlling agents for the splicing which suppresses or inhibits proximal splice site (PSS) splicing during processing of VEGF pre-mRNA transcribed from the C terminal exon 8 of the VEGF gene (see WO-A-2008/110777).

VEGFₓₓₓb and agents which promote the endogenous expression of VEGFₓₓₓb in preference to VEGFₓₓₓ in cells of a subject are encompassed by the terms “active agent” and “VEGFₓₓₓb active agent” used herein.

The VEGFₓₓₓb used in the present invention may be prepared by any suitable means. The use of agents, acting on cells to promote the endogenous expression of VEGFₓₓₓb in preference to VEGFₓₓₓ in the cells, is one possible way of preparing the VEGFₓₓₓb for use in the present invention. For further details of the agents, see WO-A-2008/110777.

The in vivo neuroprotection and neuroregeneration, or the effect of treating or preventing neuropathic and neurodegenerative disorders, is obtained in a human or animal subject, for example a human or other mammalian subject, most preferably a human subject. The term “subject” used herein will be understood in this way throughout.

The neuroprotection and neuroregeneration may be obtained in the context of a therapeutic treatment of a neuropathic or neurodegenerative disorder or condition that the subject is suffering from or to which he or she is susceptible. Alternatively, the neuroprotection and neuroregeneration may be obtained in the context of a non-therapeutic treatment of a normal subject, particularly a human subject, for example to adjust cognition or behaviour.

The term “VEGFₓₓₓb” herein includes within its scope an expression vector system which causes the expression of the VEGFₓₓₓb in a host organism, suitably the subject to be treated. Such an expression vector system suitably comprises a promoter nucleotide sequence operably associated a nucleotide sequence coding for the VEGFₓₓₓb, whereby the VEGFₓₓₓb can be expressed in the host organism under suitable conditions of transfection and incubation. Further details are provided below in the section headed “Gene Therapy”.

The VEGFₓₓₓb may for example, comprise one or more of VEGF₁₆₅b, VEGF₁₈₉b, VEGF₁₄₅b, VEGF₁₈₃b and VEGF₁₂₁b. The VEGFₓₓₓb suitably comprises recombinant VEGFₓₓₓb, preferably recombinant human VEGFₓₓₓb (rhVEGFₓₓₓb).

The VEGFₓₓₓb preferably comprises VEGF₁₆₅b, e.g. recombinant VEGF₁₆₅b, such as recombinant human VEGF₁₆₅b (rhVEGF₁₆₅b).

The VEGFₓₓₓb may, for example, consist essentially of VEGF₁₆₅b, e.g. recombinant VEGF₁₆₅b, such as recombinant human VEGF₁₆₅b (rhVEGF₁₆₅b). The VEGFₓₓₓb may, for example, consist of VEGF₁₆₅b, e.g. recombinant VEGF₁₆₅b, such as recombinant human VEGF₁₆₅b (rhVEGF₁₆₅b).

There is evidence (Example 4 below) that VEGF₁₂₁b is not as significantly effective in a neuroprotective action on retinal ganglion cells compared with control, in contrast to the significant neuroprotective effect of VEGF₁₆₅b. However, FIG. 5C nevertheless shows a certain increase in the proportion of live neurons under treatment with VEGF₁₂₁b, albeit not statistically significant or as large as the increase provided by VEGF₁₆₅b. For the time being, we cautiously include VEGF₁₂₁b in the family of VEGFₓₓₓb agents having neuroprotective and neuroregenerative activity according to the present invention, on the basis of the apparent correlation between the antiangiogenic effect of the VEGFₓₓₓb family and the unexpected neuroprotective and neuroregenerative effects, but we report this apparent lower activity in relation to retinal ganglion cells. Further work on VEGF₁₂₁b will follow.

### DETAILED DESCRIPTION OF THE INVENTION

Agents which Selectively Promote the Expression of VEGFₓₓₓb in Preference to VEGFₓₓₓ in Cells of a Subject or In Vitro

Such agents are described in the passage from page 6, line 22 to page 8, line 9 of WO-A-2008/110777, and elaborated in the remainder of WO-A-2008/110777 to the extent that favouring of DSS splicing over PSS splicing is concerned. Please refer to these passages of WO-A-2008/110777 for the discussion.

In particular, there may be mentioned Transforming Growth Factor (TGF)-b1, TGF-b R1, SRPK1 specific inhibitors (for example, SRPIN 340), T-cell intercellular antigen-1 (TIA-1), MKK3/MKK6-activatable MAP kinases (for example, p38 MAPK), Cdc20-like (Clk) family kinases Clk1/sty, Clk2, Clk3 and Clk4, the SR splicing factor SRp55, their in vivo activators, upregulators and potentiators, functionally active analogues and functionally active fragments of any of the foregoing, modified forms of any of the foregoing having a secondary functionality useful for control of their primary activity or the effects thereof, expression vector systems for expressing any of the foregoing agents in vivo, transcription/translation blocking agents which bind to the PSS of exon 8a of the pre-mRNA and/or at the region of the pre-mRNA to which a splicing regulatory protein binds, to inhibit proximal splicing (for example, morpholinos or other synthetic blocking agents, peptide conjugates, RNA binding proteins, RNA interference (RNAi) poly- and oligonucleotide blocking agents (for example dsRNA, microRNA (miRNA), siRNA), peptide nucleic acid (PNA), protein kinase C (PKC) inhibitors (for example, bisindolyl maleimide (BIM) and other mechanistically analogous PKC inhibitors, particularly inhibitors which bind at the PKC catalytic domain), or any combination thereof.

Such an expression vector system suitably comprises a promoter nucleotide sequence operably associated a nucleotide sequence coding for the agent promoting expression of VEGFₓₓₓb in preference to VEGFₓₓₓ, whereby the agent promoting expression of VEGFₓₓₓb in preference to VEGFₓₓₓ can be expressed in a host organism, suitably the subject to be treated, under suitable conditions of transfection and incubation. Further details are provided below in the section headed “Gene Therapy”.

### Conditions and Disorders to be Treated

Neuropathic disorders to be treated or prevented according to the present invention include neuropathic pain and diabetic and other neuropathies.

Neurodegenerative disorders to be treated or prevented according to the present invention include neurodegeneration of the cognitive and non-cognitive types, neuromuscular degeneration, motor-sensory neurodegeneration, ocular neurodegeneration

The activities of the proteins of the VEGFₓₓₓb family are predicted to both actively prevent and actively reverse the conditions and disorders.

Furthermore, since mild cognitive dysfunction is often associated with the normal state in certain classes of healthy people, for example the aged, persons under stress, tired or exhausted persons, the present invention is also applicable to non-therapeutic treatments of healthy people to adjust or normalise their cognitive function and behaviour, including thinking, memory, learning, concentration and reasoning.

Still further, since neuroregeneration can assist in normalising brain neural networks in subjects having psychiatric or behavioural abnormalities, whether or not these are diagnosable as one or more recognised psychiatric condition, the present invention is also applicable to therapeutic treatment of persons having psychiatric disorders and to non-therapeutic treatment of physically healthy people to adjust their cognition and behaviour towards the normal state.

For example, the present invention provides for the treatment or prevention of: pain (for example, neuropathic pain), dementia, age-related cognitive impairment, Alzheimer's disease, senile dementia of the Alzheimer's type (SDAT), Lewy body dementia, vascular dementia, Parkinson's disease, postencephalitic Parkinsonism, depression, schizophrenia, muscular dystrophy including facioscapulohumeral muscular dystrophy (FSH), Duchenne muscular dystrophy, Becker muscular dystrophy and Bruce's muscular dystrophy, Fuchs' dystrophy, myotonic dystrophy, corneal dystrophy, reflex sympathetic dystrophy syndrome (RSDSA), neurovascular dystrophy, myasthenia gravis, Lambert Eaton disease, Huntington's disease, motor neurone diseases including amyotrophic lateral sclerosis (ALS), multiple sclerosis, postural hypotension, traumatic neuropathy or neurodegeneration e.g. following stroke or following an accident (for example, traumatic head injury or spinal cord injury), Batten's disease, Cockayne syndrome, Down syndrome, corticobasal ganglionic degeneration, multiple system atrophy, cerebral atrophy, olivopontocerebellar atrophy, dentatorubral atrophy, pallidoluysian atrophy, spinobulbar atrophy, optic neuritis, sclerosing pan-encephalitis (SSPE), attention deficit disorder, post-viral encephalitis, post-poliomyelitis syndrome, Fahr's syndrome, Joubert syndrome, Guillain-Barre syndrome, lissencephaly, Moyamoya disease, neuronal migration disorders, autistic syndrome, polyglutamine disease, Niemann-Pick disease, progressive multifocal leukoencephalopathy, pseudotumor cerebri, Refsum disease, Zellweger syndrome, supranuclear palsy, Friedreich's ataxia, spinocerebellar ataxia type 2, Rhett syndrome, Shy-Drager syndrome, tuberous sclerosis, Pick's disease, chronic fatigue syndrome, neuropathies including hereditary neuropathy, diabetic neuropathy and mitotic neuropathy, prion-based neurodegeneration, including Creutzfeldt-Jakob disease (CJD), variant CJD, new variant CJD, bovine spongiform encephalopathy (BSE), GSS, FFI, kuru and Alper's syndrome, Joseph's disease, acute disseminated encephalomyelitis, arachnoiditis, vascular lesions of the central nervous system, loss of extremity neuronal function, Charcot-Marie-Tooth disease, Krabbe's disease, leukodystrophies, susceptibility to heart failure, asthma, epilepsy, auditory neurodegeneration, macular degeneration, pigmentary retinitis and glaucoma-induced optic nerve degeneration.

Generally speaking, mental disorders are not diagnosed as “psychiatric disorders” unless the associated behaviours or thoughts cause significant distress to the individual or are disruptive of his or her everyday functioning. There is therefore a borderline between diagnosable disorders and similar, but less severe or disruptive, psychological functions the treatment of which should be considered as non-therapeutic (see below).

Examples of psychiatric disorders with which the present invention is concerned include, without limitation: anxiety disorders (for example, acute stress disorder, panic disorder, agoraphobia, social phobia, specific phobia, obsessive-compulsive disorder, sexual anxiety disorders, post-traumatic stress disorder, body dysmorphic disorder and generalized anxiety disorder), childhood disorders (for example, attention-deficit hyperactivity disorder (ADHD), Asperger's disorder, autistic disorder, conduct disorder, oppositional defiant disorder, separation anxiety disorder and Tourette's disorder), eating disorders (for example, anorexia nervosa and bulimia nervosa), mood disorders (for example, depression, major depressive disorder, bipolar disorder (manic depression), seasonal affective disorder (SAD), cyclothymic disorder and dysthymic disorder), sleeping disorders, cognitive psychiatric disorders (for example, delirium, amnestic disorders), personality disorders (for example, paranoid personality disorder, schizoid personality disorder, schizotypal personality disorder, antisocial personality disorder, borderline personality disorder, histrionic personality disorder, narcissistic personality disorder, avoidant personality disorder, dependent personality disorder and obsessive-compulsive personality disorder), psychotic disorders (for example, schizophrenia, delusional disorder, brief psychotic disorder, schizophreniform disorder, schizoaffective disorder and shared psychotic disorder), and substance-related disorders (for example, alcohol dependence, amphetamine dependence, cannabis dependence, cocaine dependence, hallucinogen dependence, inhalant dependence, nicotine dependence, opioid dependence, phencyclidine dependence and sedative dependence).

The VEGFₓₓₓb active agent may, if desired, be co-administered with one or more additional active agent, for example one or more agent selected from, but not limited to, cholinesterase inhibitors, dopamine agonists (e.g. L-dopa), COMT inhibitors, MAO-B inhibitors, anti-cholinergics, acetylcholine agonists, serotonin agonists, AMPA receptor agonists, GABA receptor agonists, NMDA receptor agonists, β-adrenoceptor agonists, digoxin, dobutamine, anti-inflammatories, neurotrophic factors, statins, adenosine A2a receptor antagonists, aldose reductase inhibitors, immunomodulators, cannabinoid agonists, interferon or tricyclic anti-depressants. The term “active agent” used herein encompasses all permutations of the VEGFₓₓₓb family proteins whether singly or in any combination, with or without any one or more co-adminstered active agent of another type.

Certain of these conditions or disorders exist as so-called “spectrum” conditions and disorders, in which a wide range of combinations of symptoms, in a wide range of relative severities, present themselves. The severity of each symptom and the particular combination of symptoms will vary from individual to individual and according to the stage of progression of the disorder. In many cases of Parkinson's disease, myasthenia gravis, Lambert Eaton disease, postural hypotension and chronic fatigue syndrome, for example, cognitive dysfunction is not a primary symptom, although it may be present as one of a number of possible secondary symptoms, particularly in more advanced cases. The present invention encompasses the treatment of such conditions and disorders wherever on the spectrum they lie in any particular subject case.

The present invention may used to treat neurodegenerative diseases and disorders, neuromuscular diseases and disorders, and motor-sensory diseases and disorders where symptoms of cognitive dysfunction are either present or absent, or where any symptoms of cognitive dysfunction presented by a subject to be treated are secondary or ancillary to the symptoms of non-cognitive neurodegeneration, non-cognitive neuromuscular degeneration or motor-sensory neurodegeneration.

### Compositions and Administration

The active agent may be administered in the form of a composition comprising the active agent and any suitable additional component. The composition may, for example, be a pharmaceutical composition (medicament), a foodstuff, food supplement, beverage or beverage supplement.

According to a further aspect of the present invention, there is provided a composition comprising an effective amount of VEGFₓₓₓb active agent for use in treating or preventing, neuropathic and neurodegenerative disorders, or for use as a neuroprotective or neuroregenerative agent in vivo or in vitro (including ex vivo).

The active agent according to the present invention may be administered in the form of a composition comprising the active agent and any suitable additional component. The composition may, for example, be a pharmaceutical composition (medicament), suitably for parenteral administration (e.g. injection, implantation or infusion). The composition may alternatively, for example, be a foodstuff, food supplement, beverage or beverage supplement.

The term “pharmaceutical composition” or “medicament” in the context of this invention means a composition comprising an active agent and comprising additionally one or more pharmaceutically acceptable carriers. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations. Techniques and formulations generally may be found in Remington, The Science and Practice of Pharmacy, Mack Publishing Co., Easton, Pa., latest edition.

Liquid form preparations include solutions, suspensions, and emulsions. As an example may be mentioned water or water-propylene glycol solutions for parenteral injection. Liquid preparations can also be formulated in solution in aqueous polyethylene glycol solution.

Also included are solid form preparations which are intended to be converted, shortly before use, to liquid form preparations for either oral or parenteral administration. Such liquid forms include solutions, suspensions, and emulsions. These particular solid form preparations are most conveniently provided in unit dose form and as such are used to provide a single liquid dosage unit. Alternately, sufficient solid may be provided so that after conversion to liquid form, multiple individual liquid doses may be obtained by measuring predetermined volumes of the liquid form preparation as with a syringe, teaspoon, or other volumetric container or apparatus. The solid form preparations intended to be converted to liquid form may contain, in addition to the active material, flavourings, colourants, stabilizers, buffers, artificial and natural sweeteners, dispersants, thickeners, solubilising agents, and the like. The liquid utilized for preparing the liquid form preparation may be water, isotonic water, ethanol, glycerine, propylene glycol, and the like as well as mixtures thereof. Naturally, the liquid utilized will be chosen with regard to the route of administration, for example, liquid preparations containing large amounts of ethanol are not suitable for parenteral use.

The terms “foodstuff”, “food supplement”, “beverage” and “beverage supplement” used herein have the normal meanings for those terms, and are not restricted to pharmaceutical preparations. Other composition forms are also included within the present invention. These may, for example, include pure or substantially pure compound as such, a foodstuff precursor such as a rehydratable powder or a beverage precursor such as a powder dispersible in water, milk or other liquid.

The dosages may be varied depending upon the requirements of the patient, the severity of the condition being treated, and the compound being employed. Determination of the proper dosage for a particular situation is within the skill of the art. Generally, treatment is initiated with the smaller dosages which are less than the optimum dose of the compound. Thereafter the dosage is increased by small increments until the optimum effect under the circumstances is reached. For convenience, the total daily dosage may be divided and administered in portions during the day if desired.

### Gene Therapy

The present invention may alternatively be practiced using gene therapy. Gene therapy techniques are generally known in this art, and the present invention may suitably be put into practice in these generally known ways. The following discussion provides further outline explanation.

The gene therapies are broadly classified into two categories, i.e., in vivo and in vitro therapies. The in vivo gene therapy comprises introducing a therapeutic gene directly into the body, and the in vitro gene therapy comprises culturing a target cell in vitro, introducing a gene into the cell, and then, introducing the genetically modified cell into the body.

The gene transfer technologies are broadly divided into a viral vector-based transfer method using virus as a carrier, a non-viral delivery method using synthetic phospholipid or synthetic cationic polymer, and a physical method, such as electroporation or introducing a gene by applying temporary electrical stimulation to a cell membrane.

Among the gene transfer technologies, the viral vector-based transfer method is considered to be preferable for the gene therapy because the transfer of a genetic factor can be efficiently made with a vector with the loss of a portion or whole of replicative ability, which has a gene substituted a therapeutic gene. Examples of virus used as the virus carrier or vector include RNA virus vectors (retrovirus vectors, lentivirus vector, etc.), and DNA virus vectors (adenovirus vectors, adeno-associated virus vectors, etc.). In addition, its examples include herpes simplex viral vectors, alpha viral vectors, etc. Among them, retrovirus and adenovirus vectors are particularly actively studied.

The characteristics of retrovirus acting to integrate into the genome of host cells are that it is harmless to the human body, but can inhibit the function of normal cells upon integration. Also, it infects various cells, proliferates fast, can receive about 1-7 kb of foreign genes, and is capable of producing replication-deficient virus. However, it has disadvantages in that it is hard to infect cells after mitosis, it is difficult to transfer a gene in vivo, and the somatic cell tissue is needed to proliferate always in vitro. In addition, since it can be integrated into a proto-oncogene, it has the risk of mutation and can cause cell necrosis.

Meanwhile, adenovirus has various advantages for use as a cloning vector; it has moderate size, can be replicated within a cell nucleus, and is clinically nontoxic. Also, it is stable even when inserted with a foreign gene, and does not cause the rearrangement or loss of genes, can transform eucaryotes, and is stably expressed at a high level even when it is integrated into the chromosome of host cells. Good host cells for adenovirus are cells of causing human hematosis, lymphoma and myeloma. However, these cells are difficult to proliferate because they are linear DNAs. Also, it is not easy infected virus to be recovered, and they have low virus infection rate. Also, the expression of a transferred gene is the highest after 1-2 weeks, and in some cells, the expression is kept only for about 3-4 weeks. In addition, these have the problem of high immune antigenicity.

Adeno-associated virus (AAV) can overcome the above-described problems and at the same time, has many advantages for use as a gene therapeutic agent and thus is recently considered to be preferable. AAV, which is single-strand provirus, requires an assistant virus for replication, and the AAV genome is 4,680 bp in size and can be inserted into any site of chromosome 19 of infected cells. A trans-gene is inserted into plasmid DNA linked with 145 bp of each of two inverted terminal repeat sequence (ITR) and a signal sequence. This gene is transfected with another plasmid DNA expressing AAV rep and cap genes, and adenovirus is added as an assistant virus. AAV has advantages in that the range of its host cells to be transferred with a gene is wide, immune side effects due to repeated administration are little, and the gene expression time is long. Furthermore, it is stable even when the AAV genome is integrated into the chromosome of a host cell, and it does not cause the modification or rearrangement of gene expression in host cells. Since an AAV vector containing a CFTR gene was approved by NIH for the treatment of cystic fibrosis in 1994, it has been used for the clinical treatment of various diseases. An AAV vector containing a factor IX gene, which is a blood coagulation factor, is used for the treatment of hemophilia B, and the development of a therapeutic agent for hemophilia A with the AAV vector is currently being conducted. Also, AAV vectors containing various kinds of anticancer genes were certified for use as tumor vaccines.

Gene therapy, which is a method of treating diseases by gene transfer and expression, is used to adjust a certain gene, unlike the drug therapy. The ultimate purpose of the gene therapy is to obtain useful therapeutic effects by genetically modifying a living gene. The gene therapy has various advantages, such as the accurate transfer of a genetic factor into a disease site, the complete decomposition of the genetic factor in vivo, the absence of toxicity and immune antigenicity, and the long-term stable expression of the genetic factor and thus is spotlighted in connection with the present invention as a potentially suitable route of treatment.

In general, reference herein to the presence of one of a specified group of compounds, for example VEGF b, includes within its scope the presence of a mixture of two or more of such compounds.

### “Treating or Preventing”

The expression “treating or preventing” and analogous terms used herein refers to all forms of healthcare intended to remove or avoid the disorder or to relieve its symptoms, including preventive, curative and palliative care, as judged according to any of the tests available according to the prevailing medical and psychiatric practice. An intervention that aims with reasonable expectation to achieve a particular result but does not always do so is included within the expression “treating or preventing”. An intervention that succeeds in slowing or halting progression of a disorder is included within the expression “treating or preventing”.

Certain neurological and psychiatric disorders are considered as “spectrum” conditions, in which individuals may exhibit some or all of a range of possible symptoms, or may exhibit only a mild form of the disorder. Furthermore, many neurological and psychiatric conditions are progressive, starting with relatively mildly abnormal symptoms and progressing to more severely abnormal symptoms. The present invention includes the treatment and prevention of all neurological and psychiatric conditions of whatever type and stage.

“Susceptible to”

The expression “susceptible to” and analogous terms used herein refers particularly to individuals at a higher than normal risk of developing a medical or psychiatric disorder, or a personality change, as assessed using the known risk factors for the individual or disorder. Such individuals may, for example, be categorised as having a substantial risk of developing one or more particular disorders or personality changes, to the extent that medication would be prescribed and/or special dietary, lifestyle or similar recommendations would be made to that individual.

### “Non-Therapeutic Method”

The expression “non-therapeutic method” used herein refers particularly to an intervention performed on an individual who is neurologically or psychologically within the normal range, to normalise or enhance or improve a function of the neurological or psychological kind. A neurological function that may suitably be treated non-therapeutically may include, for example, cognition (including thinking, reasoning, memory, recall, imagining and learning), concentration and attention, particularly towards the milder end of the scale of conditions, and mild abnormal behavioural or personality traits. A psychological function that may suitably be treated non-therapeutically may include, for example, human behaviour, mood, personality and social function, for example grief, anxiety, depression, moodiness, moroseness, teenage moods, disrupted sleep patterns, vivid dreaming, nightmares, and sleepwalking.

There is a borderline between diagnosable neurological and psychiatric disorders and (non-diagnosable) neurological and psychological functions within the normal range. Therefore, in addition to the examples of neurological and psychological functions give above that are treatable according to the non-therapeutic methods of the present invention, mild forms of neurological and psychiatric disorders, that are non-diagnosable because the associated behaviours or thoughts do not cause significant distress to the individual or are not disruptive of his or her everyday functioning, are also to be considered as conditions treatable non-therapeutically according to the present invention.

### “Normalise”

The expression “normalise” and analogous terms used herein refers particularly to a physiological adjustment towards a condition characteristic of general normal neurological or psychiatric health, whether or not a condition is actually reached that would be characterised as normal.

### Mammals

Besides being useful for human treatment, the present invention is also useful in a range of mammals, which can also be affected by neurological and psychological/psychiatric conditions. Such mammals include non-human primates (e.g. apes, monkeys and lemurs), for example in zoos, companion animals such as cats or dogs, working and sporting animals such as dogs, horses and ponies, farm animals, for example pigs, sheep, goats, deer, oxen and cattle, and laboratory animals such as rodents (e.g. rabbits, rats, mice, hamsters, gerbils or guinea pigs).

Where the disorder or function to be treated is exclusive to humans, then it will be understood that the mammal to be treated is a human. The same applies respectively to any other mammalian species if the disorder or function to be treated is exclusive to that species.

### BRIEF DESCRIPTION OF THE DRAWINGS

In order to illustrate the invention further by way of non-limiting example, reference will now be made to the accompanying drawings and to the Examples which follow. In the drawings:

FIG. 1 (a) to (d) illustrates the expression of VEGF₁₆₅b in developing and adult human neuronal tissues. Immunohistochemistry of (A) embryonic (12 week old) and (B) adult neuronal tissue. Brown staining was seen in the spinal cord, particularly in the ventral horn and the dorsal root ganglion cells, and the developing brain. In adult tissues neurons and blood vessels were seen in cortex and in hippocampus. Protein extracted from adult human DRG showed significant expression of VEGF₁₆₅b. rVEGF=50 ng recombinant VEGF D. Measurement of VEGF in protein extract demonstrates that VEGF165b forms 71% of the total VEGF in the DRG;

FIG. 2 (a) to (d) illustrates the alteration in VEGF₁₆₅b expression in DRG neurons in a rat model of nerve injury. Rat sciatic nerve was dissected and cut, and the animal allowed to recover for 24 hours. The animals were then killed and L4 DRG innervated by the sciatic was then excised and processed for Western blotting or immunohistochemistry. A. Western blot showing increased expression of VEGF₁₆₅b in ipsilateral DRG compared with contralateral and normal (untreated) DRG. B. Sections through the center of the DRG showed clear staining of DRG neurogs (arrows). Measurement of neuronal areas, showed that there was a significant decrease in the number of small neurons (more likely to be nociceptors) staining for VEGF₁₆₅b (p<0.05). In contrast medium sized and larger neurons (motor neurons) increased their VEGF₁₆₅b expression after nerve injury. B. This resulted in an increase in the mean area of nerves expressing VEGF₁₆₅b in nerve injured compared to both the contralateral side in these rats (p<0.01), but also compared with naïve, untreated rats (p<0.001);

FIGS. 3 (a) and (b) illustrates the effect of VEGF₁₆₅b on survival of neurons Brains were dissected from freshly killed neonatal mice. Slices of hippocampus were placed in chambers and treated with 3 mM glutamate either in saline, with 100 nM galanin (Gal) or 10 nM VEGF₁₆₅b. Cells were then fixed and stained with propidium iodide and dead cells counted by fluorescence microscopical analysis. 10 nM VEGF₁₆₅b reduced glutamate induced cytotoxicity by 50% and 27% in CA1 and CA3 neurons respectively;

FIG. 4 illustrates the effect of VEGF₁₆₅b on DRG neurite outgrowths. Rat DRG were excised, and neurons cultured for 8 hours. Cells were then treated with 1 nM VEGF₁₆₅b for 24 hours and neurite outgrowths measured by light microscopy;

FIG. 5 (a) to (d) illustrates that VEGF₁₆₅b is neuroprotective to retinal ganglion cells in vivo. Retinal ganglion cells were retrogradly labeled with FluorGold using stereostatic injection into in the superior colliculi. 7 days later, 10 ng rhVEGF₁₆₅b, or HBSS (controls) was injected into the left vitreous. 24 hours after injection Retinal ischemia-reperfusion injury was induced in the by elevation of the intraocular pressure through saline perfusion into one anterior chamber. 13 days later, the animals were killed, and both retinae flat mounted and imaged using fluorescence microscopy. A. Pseudocolored fluorescent images of retinal cells in an example of (I) the contralateral (non ischemic) eye, (ii) control eye injected with HBSS, or (iii) eye injected with VEGF₁₆₅b. B. Retinal ganglion cell counts were significantly lower in the ischemic group compared with the non ischemic group. In contrast VEGF₁₆₅b, injected retinas had more viable retinal ganglion cells. P<0.001, ANOVA Boneferroni Post-hoc test). C. The ratio of retinal ganglion cells per field in the ischemic compared with the non ischemic contralateral eye was calculated. Control (HBSS) treated eyes had significantly lower ratio of live cells than VEGF₁₆₅b treated eyes. D. Active caspase 3 (red) immunofluorescence staining of eye cross-sections. Nuclei stained blue with Hoechst 33258. After 24 h ischemia-reperfusion injury, active caspase 3 was dominantly present in the retinal ganglion cell/neurofiber layer and inner nuclear layer in the retina. The numbers of active caspase 3 positive cells were lower in VEGF₁₆₅b treated eye than HBSS treated one. (Scale=200 μm). *=P<0.05**=p<0.01, ***=p<0.001 compared with VEGF165b++=p<0.01 compared with HBSS; and

FIG. 6 illustrates the effect of VEGF inhibition to reduce (a) mechanical and (b) thermal pain thresholds in DRG neurons. Mechanical and thermal nociceptive withdrawals were measured in male C57Bl6 mice prior to i.p. injection of either vehicle (saline) or 6 μg/g anti-mouse VEGF antibody. Mechanical and thermal nociceptive thresholds were then measured 3 (mechanical) and 6 (thermal) hours after injection. Anti-VEGF antibody injection results in a small mechanical allodynia, and a larger thermal hyperalgesia. *p<0.05, **p<0.01 anti-VEGF compared to baseline and vehicle as shown, Kruskal-Wallis+Dunn's.

### DETAILED DESCRIPTION OF THE DRAWINGS AND EXAMPLES

### Example 1

### VEGF₁₆₅b Expression in Developing and Adult Human Neuronal Tissues

### VEGF₁₆₅b is Endogenously Expressed in the Human Nervous System.

To determine whether VEGF₁₆₅b was expressed in the nervous system, human embryonic and adult tissues were stained using the VEGFₓₓₓb specific antibody MAB3045 (available from R&D Systems, UK) or biotinylated clone 264610/10 (available from R&D Systems, UK) which has been extensively characterised and detects human VEGFₓₓₓb isoforms, but does not detect VEGFₓₓₓ isoforms (Varey et al Br J. Cancer. In press, 2008). ELISA and Western blotting of human proteins was carried out as previously described (Woolard J., Cancer Res 64: 7822-7835, 2004). Total VEGF ELISA was carried out using the commercial R&D quantikine VEGF ELISA kit (available from R&D Systems, UK).

Three human female fetuses of 10 and 12 weeks pregnancy were obtained with local ethics committee approval (Leiden).

Human brain samples were taken from the human brain bank at Frenchay Hospital, Bristol, UK, with local ethics committee approval (North Bristol Ethics Committee).

Determination of VEGF expression in rat DRG was carried out by immunohistochemistry using a commercially available R&D antibody, clone 264610/1 (available from R&D Systems, UK). Western blotting was carried out in the same manner as for human proteins.

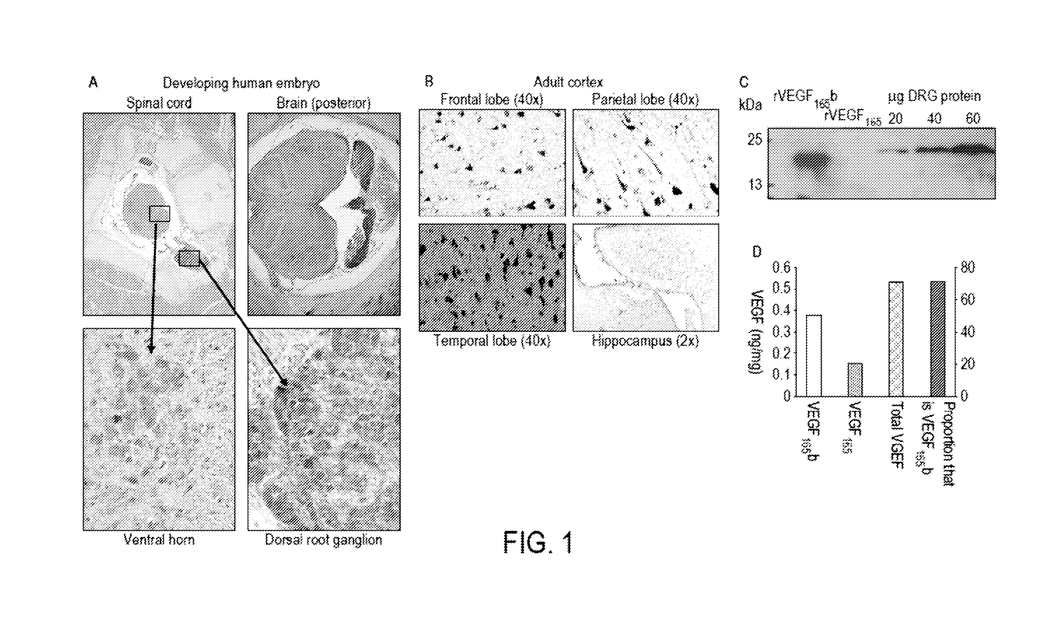

FIG. 1A shows that staining was intense in the developing brain and spinal cord of ten week old human embryos. In particular, the ventral horn and the dorsal root ganglia had intense staining of neuronal cell bodies, as well as weaker staining of the nerve fibre tracts. The negative control matched isotype IgG was clean.

To determine if expression was also seen in adult brain and spinal cords, sections of normal brain tissue were stained. Strong and distinct staining was seen in neurons of the cortex, through layers II-VI, with less expression in layer I in all sections of the cortex examined (parietal temporal and frontal lobes). The subcortical layer showed weaker expression in nerve fibre tracts. Expression was also seen in specific neurons of the hypothalamus (FIG. 1B).

To determine expression in dorsal root ganglia, protein from DRG was subjected to Western blotting and ELISA. Western blotting showed that recombinant human VEGF₁₆₅b (21 kDa, as determined by mass spectroscopy) was detected by the VEGF₁₆₅b antibody, but not VEGF₁₆₅. In human DRG bands were seen at 23 kDa, the predicted size for endogenous, fully glycosylated VEGF₁₆₅b (FIG. 1C).

Moreover, ELISA showed that VEGF₁₆₅b expression accounted for a significant proportion of the VEGF present in DRG, amounting to 71% of total VEGF (FIG. 1D).

To determine whether VEGF₁₆₅b expression was altered in sensory nerves during nerve injury, the following experiment was performed. The rat DRG neuron was used as a model of nerve injury.

Rat sciatic nerve was dissected and cut, and the animal allowed to recover for 24 hours. The animals were then killed and L4 DRG innervated by the sciatic was then excised and processed for Western blotting or immunohistochemistry.

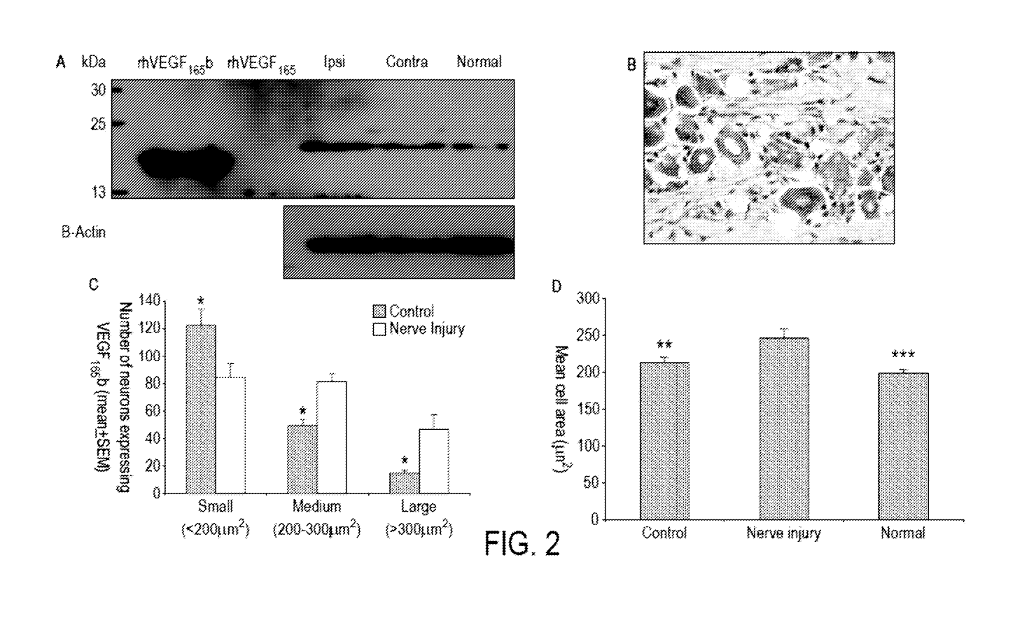

FIG. 2A is the Western blot showing increased expression of VEGF₁₆₅b in ipsilateral DRG compared with contralateral and normal (untreated) DRG. FIG. 2B shows sections through the centre of the DRG showed clear staining of DRG neurons (arrows).

Western blotting indicated that there was an upregulation of VEGF₁₆₅b in the DRG ipsilateral to the nerve section relative to both the contralateral expression and untreated rat DRG. To determine the location of this change in expression, immunohistochemistry was performed. VEGF₁₆₅b expression was seen in a subset of soma in the DRG (FIG. 2B). Measurement of neuronal areas showed that there was a significant decrease in the number of small (less than 200 μm²) neurons (more likely to be nociceptors) staining for VEGF₁₆₅b (p<0.05). In contrast medium sized and larger neurons increased their VEGF₁₆₅b expression after nerve injury (FIG. 2C). This resulted in an increase in the mean area of nerves expressing VEGF₁₆₅b in nerve injured compared to both the contralateral side in these rats (p<0.01), but also compared with naïve, untreated rats (p<0.001, FIG. 2D).

### Example 2

### Effect of VEGF₁₆₅b on Neuronal Cell Death

### VEGF₁₆₅b is a Survival Factor for Neurons In Vivo

To determine whether VEGF₁₆₅b was able to act directly on neurons as a survival factor, we examined the effect of VEGF₁₆₅b on cytotoxicity of hippocampal neurons.

Brains were dissected from freshly killed neonatal mice. Slices of hippocampus were placed in chambers and treated with the neurocytotoxic agent glutamate (3 mM) either in saline, with 100 nM galanin (Gal) positive control, or 10 nM VEGF₁₆₅b.

Cells were then fixed and stained with propidium iodide and dead cells were counted by fluorescence microscopical analysis.

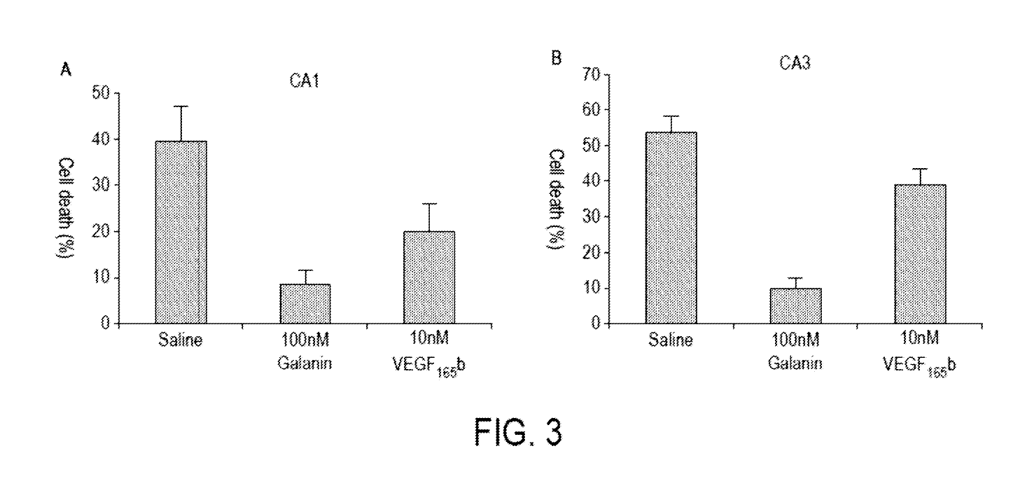

Both CA1 neurons (FIG. 3A) and CA3 neurons (FIG. 3B) showed an inhibition of cytotoxicity when treated with 10 nM VEGF₁₆₅b, although not as significantly as 100 nM Galanin 10 nM VEGF₁₆₅b reduced glutamate-induced cytotoxicity by 50% and 27% in CA1 and CA3 neurons respectively.

### Example 3

### Effect of VEGF₁₆₅b on Neurite Outgrowth

### VEGF₁₆₅b Reduced Glutamate-Induced Cytotoxicity in Brain Neurons

To determine whether VEGF₁₆₅b could act to induce neurite outgrowth from DRG, rat DRG cultures were set up and treated with 1 nM VEGF₁₆₅b or control medium, The neurons were cultured for 8 hours. Cells were then treated with 1 nM VEGF₁₆₅b for 24 hours and neurite outgrowths were measured by light microscopy.

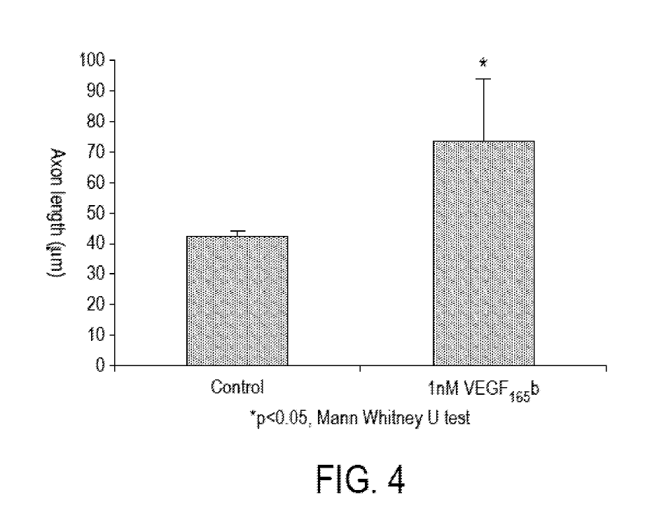

The results are shown in FIG. 4. Culturing with 1 nM VEGF₁₆₅b results in significantly longer neurite outgrowths, although there was no difference in cell numbers between treated and control cultures.

### Example 4

### Effect of VEGF₁₆₅b on Neuroprotection

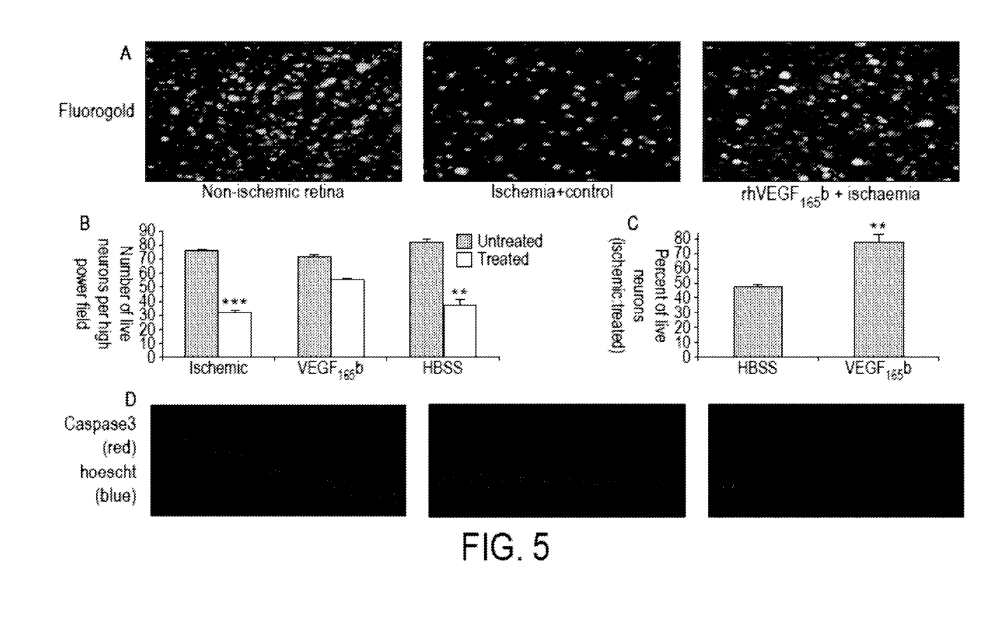

To determine whether VEGF₁₆₅b was neuroprotective in vivo we used a system of local application to a ganglion cell population after injury—the ocular neuronal ischemia model. Rats were injected into the supracollicular area with fluorescent retrograde tracer, ischemia induced by increased intraocular pressure and treated with VEGF₁₆₅b or control solution (HBSS). Retinas were then excised and mounted and live neurons counted. FIG. 5A shows that the number of fluorescently labelled neurons was reduced by ischemia, but VEGF₁₆₅b treatment resulted in an increase in fluorescent labelling.

Retinal ganglion cells were retrogradely labeled with Fluor-Gold (4% in PBS, Fluorchrome Inc., Denver, Colo.) as previously described (Selles-Navarro et al, Invest Ophthalmol Vis. Sci. 1996 September; 37 (10): 2002-14). 35 Wistar rats were employed in this study. Six holes were drilled to the exposed scull of anaesthetized and immobilized rats in a stereotaxic apparatus; Fluor-Gold was injected with a 30 G needle bilaterally into the superior colliculi, 0.6 μl at 4.2 mm and 0.7 μl at 4.7 mm depth. 60 seconds after injection, the needle was withdrawn, and the skin sutured. The dye is picked up by the axon terminals of the RGCs and bilaterally transported. The somas of RGC are thereby retrogradely labeled and maintain this marker for at least three weeks without significant fading or leakage.

Seven days after RGC labeling, the left eye of the rat received an intravitreous injection of either 10 ng rhVEGF₁₆₅b in HBSS (250 fmol, R&D Systems, UK), 1 ng rhVEGF₁₂₁b (250 fmol, R&D Systems, UK) or HBSS and the right eye was left untreated. Retinal ischemia was induced through a 0.9% NaCl infusion into the anterior chamber in the left eye as previously described. Pupils were dilated with 5% phenylephrine-HCl and 5% tropicamid eye drops. A 30 G needle was inserted via the corneal limbus into the anterior chamber using a surgical microscopical stereotactic setup (S5, Zeiss), and connected to a 140-150 mmHg (19.95 kPa) saline infusion. The cessation of retinal blood flow was visibly monitored in fundoscopy. After 60 mins ischemia, reperfusion followed as the needle was withdrawn.

Thirteen days after the ischemia-reperfusion injury, the rats were sacrificed and the eyes were fixed in 4% PFA for 4 hours at 4° C., washed in PBS over night and dissected. Retinas were flat mounted in anti-fading medium (Fluor Mount, DAKO) by four radial cuts and thereby divided into four quarters. Specimens were examined with fluorescence microscopy (Zeiss, Axion) equipped with a digital camera. In each quarter of the retina, a random image was taken in the central region around the optic disc, one in the peripheral and one in the intermedial region. FIG. 5A shows fluorescent images of retinal cells in examples of (i) the contralateral (non-ischaemic) eye, (ii) the control eye injected with HBSS, and (iii) the eye injected with VEGF₁₆₅b. A trained observer counted the numbers of positively labeled ganglion cells in a single blinded fashion.

Four animals in each group were sacrificed 24 hours after ischemic phase finished. The eye was removed and immersed into 4% paraformadihyde (PFA) at 4° C. overnight, the cornea and lens were then removed, and the eyecups washed in 30% sucrose in 0.5 mM phosphate buffer at 4° C. overnight. The eyecups were then embedded in optimum temperature compound (Leica) and stored at −80° C. until use. 6 μm cross-sections were cut for the immunofluorescence staining After the sections were rehydrated in phosphate buffered saline (PBS), primary antibodies were incubated in PBS-T (0.5% TritonX 100, 0.25% BSA) with normal serum, either overnight or for one hour in a humid chamber at room temperature. The following primary antibodies were used: polyclonal rabbit anti-mouse active caspase 3 with cross-reactivity (1:2000, R&D Systems), polyclonal rabbit anti-GFAP (1:200, NeoMarker), as well as biotinized isolectin B4 (1:100, Vector Laboratories). Sections were washed twice in PBS, before they were incubated with secondary antibodies in PBS-T. Goat anti-rabbit IgG or streptavidin-conjugated Alexa Fluor 488 and 594 were used (Molecular Probes). After wash, the sections were mounted in anti-fading medium (Fluor Mount, DAKO). Samples were examined with fluorescence microscopy (Zeiss, Axion) equipped with a digital camera. Images were processed in Adobe Photoshop.

FIGS. 5B and C show the results of the fluorescence microscopy on the excised retinas.

The retinal ganglion cell counts were significantly lower in the ischemic group compared with the non-ischemic group. In contrast, VEGF₁₆₅b, but not VEGF₁₂₁b, injected retinas had more viable retinal ganglion cells (P<0.001, ANOVA Boneferroni Post-hoc test) (FIG. 5B). The ratio of retinal ganglion cells per field in the ischemic compared with the non-ischemic contralateral eye was calculated. Control (HBSS) treated eyes had a significantly lower ratio of live cells than VEGF₁₆₅b treated eyes. VEGF₁₂₁b did not significantly increase the ratio compared with control. (*=P<0.05**=p<0.01***=p<0.001 compared with VEGF₁₆₅b ++=p<0.01 compared with HBSS NS=not significantly different from HBSS) (FIG. 5C). Activated caspase 3 staining revealed a reduction in apoptotic cells in the retinal ganglion cell layer (fewer red cells in FIG. 5D), indicating that VEGF₁₆₅b inhibited apoptosis in the retinal neurons.

### Example 5

### Effect of VEGF₁₆₅b on Pain Threshold

To determine the effect of VEGF₁₆₅b on pain thresholds, 6 μg/g anti-mouse VEGF antibody in saline were injected i.p. into the neurons of the dorsal root ganglion (DRG) of male C57BI6 mice, where the predominantly expressed VEGF isoform is VEGF₁₆₅b. Mechanical and thermal nociceptive thresholds were then measured 3 (mechanical) and 6 (thermal) hours after injection, as latency to withdrawal after stimulus, and expressed as percentage of control where control=the same animal before injection.

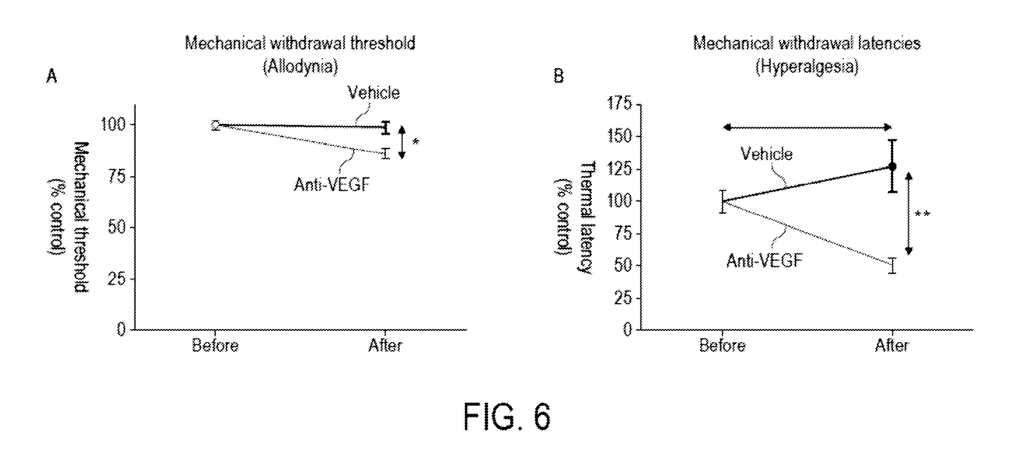

The results are shown in FIGS. 6A (mechanical withdrawal thresholds—allodynia) and 6B (thermal withdrawal latencies—hyperalgesia).

Anti-VEGF antibody inhibits VEGF and results in a small mechanical allodynia (FIG. 6A; *=p<0.05) and a larger thermal hyperalgesia (FIG. 6B; **=p<0.01) anti-VEGF compared to baseline and vehicle as shown (Kruskal-Wallis+Dunn's).

The data show that removing all VEGFs in the DRG neurons results in a greater sensitivity to pain vs. vehicle alone (i.e. lowers the pain threshold). From this it appears that, as the vast majority of VEGF in this tissue is VEGFₓₓₓb, and particularly VEGF₁₆₅b, this isoform is acting to reduce pain sensitivity (increasing the pain threshold). FIG. 6B indicates that the vehicle alone (saline) increases thermal latency but this is not significant and may be due to a degree of animal learning.

### INDUSTRIAL APPLICABILITY

The present invention provides a new family of active agents for use against neuropathy and neurodegeneration, for example in CNS, peripheral and motor-sensory neurons.

The neuroprotective and neuroregenerative activity of the VEGFₓₓₓb family of proteins, and particularly VEGF₁₆₅b, is unexpected in view of the known properties of the proteins.

This finding opens up many new therapeutic and other treatments of human and animal subjects suffering from or susceptible to a range of neuropathic and neurodegenerative conditions and disorders.

1-13. (canceled)

14. A method for treating or preventing a neuropathic or a neurodegenerative disorder in a subject suffering from or susceptible to a neuropathic or a neurodegenerative disorder, or of obtaining neuroprotection or neuroregeneration in vivo or in vitro, the method comprising:
administering to neurons, in vivo or in vitro, an effective amount of an agent which selectively promotes the expression of VEGFₓₓₓb in preference to VEGFₓₓₓ in cells of a subject or in vitro,
wherein the agent which selectively promotes the expression of VEGFₓₓₓb in preference to VEGFₓₓₓ is selected from
SRPK1-specific inhibitors, SRPK2-specific inhibitors, SRPK1 and SRPK2-specific inhibitors, T-cell intercellular antigen-1 (TIA-1), MKK3/MKK6-activatable MAP kinases, Clk1/sty, Clk2, Clk3 and Clk4;
an expression vector system for expressing any of the foregoing agents in vivo;
an expression vector system which causes the expression of the VEGFₓₓₓb in a host organism; and
any combination thereof.

15. The method according to claim 14, wherein the VEGFₓₓₓb comprises one or more of VEGF₁₆₅b, VEGF₁₈₉b, VEGF₁₄₅b, VEGF₁₈₃b and VEGF₁₂₁b.

16. The method according to claim 14, wherein the VEGFₓₓₓb comprises VEGF₁₆₅b.

17. The method according to claim 14, wherein the agent which selectively promotes the expression of VEGFₓₓₓb in preference to VEGFₓₓₓ is selected from SRPK1-specific inhibitors, SRPK2-specific inhibitors, SRPK1 and SRPK2-specific inhibitors, T-cell intercellular antigen-1 (TIA-1), MKK3/MKK6-activatable MAP kinases, Clk1/sty, Clk2, Clk3 and Clk4.

18. The method according to claim 14, further comprising co-administration of at least one agent selected from the group consisting of cholinesterase inhibitors, dopamine agonists, COMT inhibitors, MAO-B inhibitors, anti-cholinergics, acetylcholine agonists, serotonin agonists, AMPA receptor agonists, GABA receptor agonists, NMDA receptor agonists, β-adrenoceptor agonists, digoxin, dobutamine, anti-inflammatories, neurotrophic factors, statins, adenosine A2a receptor antagonists, aldose reductase inhibitors, immunomodulators, cannabinoid agonists, interferon and tricyclic anti-depressants.

19. The method according to claim 14, wherein the neuropathic or neurodegenerative disorder is selected from pain, dementia, Alzheimer's disease and Parkinson's disease.
